(19) United States
(12) Patent Application Publication
(10) Pub. No.: US 2024/0027555 A1
(43) Pub. Date: Jan. 25, 2024

(54) MAGNETIC RESONANCE IMAGING APPARATUS

(71) Applicant: CANON MEDICAL SYSTEMS CORPORATION, Otawara-shi (JP)

(72) Inventors: Akihiro ODA, Otawara (JP); Kazunori IKEZAWA, Otawara (JP); Yoshikatsu ITADA, Yaita (JP); Kazuo GUNJI, Otawara (JP); Akiko MIYATA, Sakura (JP); Takeshi ISHIMOTO, Arakawa (JP); Shinsuke KOMAKI, Otawara (JP)

(73) Assignee: CANON MEDICAL SYSTEMS CORPORATION, Otawara-shi (JP)

(21) Appl. No.: 18/345,448

(22) Filed: Jun. 30, 2023

(30) Foreign Application Priority Data
Jul. 19, 2022 (JP) 2022-115107

(51) Int. Cl. G01R 33/483; G01R 33/48

(52) U.S. Cl. CPC G01R 33/483; G01R 33/4818

(57) ABSTRACT

A magnetic resonance imaging apparatus according to the present embodiment comprises processing circuitry configured to set a region of interest for a locator image, automatically set an imaging condition based on the region of interest, and cause a sequence control circuitry to perform imaging based on the imaging condition, the sequence control circuitry performing the imaging.

### CROSS REFERENCE TO RELATED APPLICATIONS

This application is based upon and claims the benefit of priority from the prior Japanese Patent Application No. 2022-115107, filed on Jul. 19, 2022, the entire contents of which are incorporated herein by reference.

### FIELD

Embodiments described in the present specification and drawings relate generally to a magnetic resonance imaging apparatus.

### BACKGROUND

Conventionally, in the medical field, a magnetic resonance imaging (MRI) apparatus that excites a nuclear spin of a subject placed in a static magnetic field with a radio frequency (RF) signal of a Larmor frequency and reconstructs a magnetic resonance (MR) signal generated from the subject with the excitation to generate an image has been used.

In the case of imaging using such a magnetic resonance imaging apparatus, before performing main imaging for collecting images mainly used for diagnosis, an operator performs positioning imaging for collecting a positioning image, which is a positioning image referred to when imaging conditions are set. Then, the operator sets imaging conditions for main imaging based on the positioning image collected by the positioning imaging. Hereinafter, the positioning imaging is referred to as locator imaging, and the positioning image is referred to as a locator image.

However, since the operator needs to manually set the imaging conditions for the main imaging in accordance with a region of interest while referring to the locator image, a burden on the operator is large, and it takes time to set the imaging conditions for the main imaging. Therefore, in imaging using the magnetic resonance imaging apparatus, it is desired to reduce the time required for setting the imaging conditions while reducing the burden on the operator.

### BRIEF DESCRIPTION OF THE DRAWINGS

FIG. 1 is a block diagram illustrating a configuration example of a magnetic resonance imaging apparatus according to a first embodiment;

FIG. 2 is a flowchart illustrating the contents of output image generation processing performed by the magnetic resonance imaging apparatus according to the first embodiment;

FIG. 3 is a diagram illustrating an example of an imaging condition setting screen for setting imaging conditions displayed on a display of the magnetic resonance imaging apparatus according to the first embodiment;

FIG. 4 is a diagram illustrating an example of a locator image generated in the magnetic resonance imaging apparatus according to the first embodiment;

FIG. 5 is a diagram illustrating an example of an imaging plan screen displayed on the display of the magnetic resonance imaging apparatus according to the first embodiment;

FIG. 6 is a diagram illustrating an example of a locator image displayed on the imaging plan screen in the magnetic resonance imaging apparatus according to the first embodiment;

FIG. 7 is a diagram illustrating an example of a figure indicating a region of interest superimposed upon receiving an input operation from an operator on a locator image displayed on the imaging plan screen in the magnetic resonance imaging apparatus according to the first embodiment;

FIG. 8 is a diagram illustrating an example of a region of interest set in accordance with an input operation, from an operator, for superimposing a figure indicating the region of interest on a locator image in the magnetic resonance imaging apparatus according to the present embodiment;

FIG. 9 is a diagram illustrating an example of an output image information setting screen displayed on the display of the magnetic resonance imaging apparatus according to the first embodiment;

FIG. 10 is a diagram illustrating an example of a case of setting a FOV of an image in main imaging in the magnetic resonance imaging apparatus according to the first embodiment;

FIG. 11 is a diagram illustrating an example of imaging conditions for main imaging set based on a region of interest set for a locator image in the magnetic resonance imaging apparatus according to the first embodiment;

FIG. 12 is a diagram illustrating an example of a reconstructed image generated by reconstruction processing in the magnetic resonance imaging apparatus according to the first embodiment;

FIG. 13 is a diagram illustrating an example of a reconstructed image to which a region of interest is applied in the magnetic resonance imaging apparatus according to the first embodiment;

FIG. 14 is a diagram illustrating an example of an output image generated in the magnetic resonance imaging apparatus according to the first embodiment;

FIG. 15 is a flowchart illustrating the contents of output image generation processing performed by a magnetic resonance imaging apparatus according to a second embodiment;

FIG. 16 is a diagram illustrating an example of a locator image generated in the magnetic resonance imaging apparatus according to the second embodiment;

FIG. 17 is a diagram illustrating an example of a locator image displayed on an imaging plan screen in the magnetic resonance imaging apparatus according to the second embodiment;

FIG. 18 is a diagram illustrating an example of a region of interest set by receiving an input operation for setting a pixel value with respect to a locator image displayed on the imaging plan screen in the magnetic resonance imaging apparatus according to the second embodiment;

FIG. 19 is a diagram illustrating an example of a plurality of regions of interest set for one locator image in the magnetic resonance imaging apparatus according to the second embodiment;

FIG. 20 is a diagram illustrating an example of an output image information setting screen displayed on a display in the magnetic resonance imaging apparatus according to the second embodiment;

FIG. 21 is a diagram illustrating an example of a case of setting a FOV of an image in main imaging in the magnetic resonance imaging apparatus according to the second embodiment;

FIG. 22 is a diagram illustrating an example of imaging conditions set based on a region of interest set for a locator image in the magnetic resonance imaging apparatus according to the second embodiment;

FIG. 23 is a diagram illustrating an example of a reconstructed image generated by reconstruction processing in the magnetic resonance imaging apparatus according to the second embodiment; and

FIG. 24 is a diagram illustrating an example of an output image generated in the magnetic resonance imaging apparatus according to the second embodiment.

### DETAILED DESCRIPTION

Hereinafter, respective embodiments of the magnetic resonance imaging apparatus will be described with reference to the accompanying drawings. In the embodiments below, the same reference signs are given for identical components in terms of configuration and function, and duplicate description is omitted.

### First Embodiment

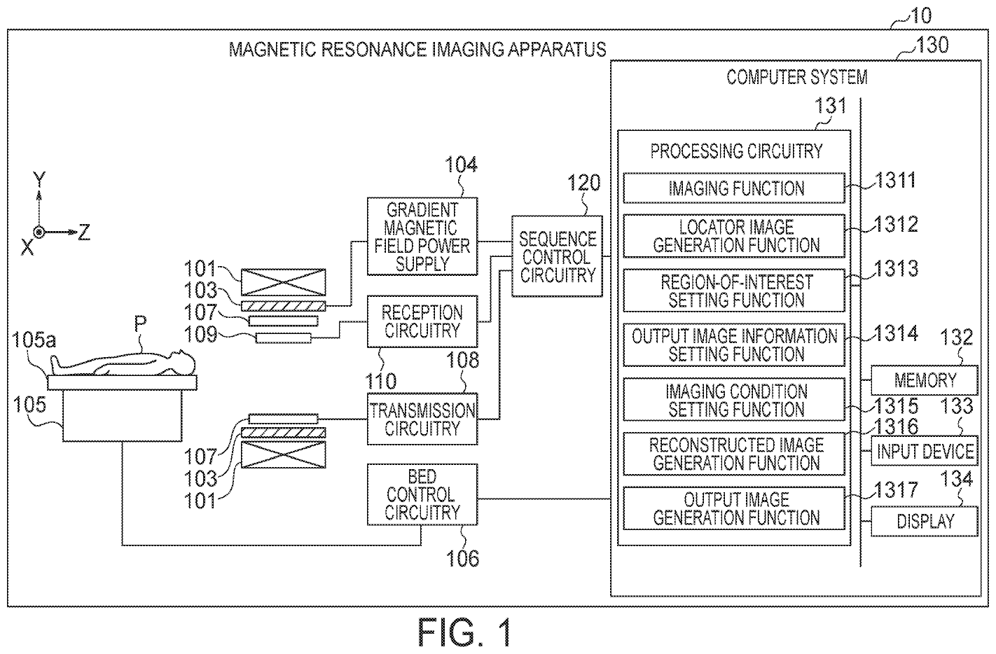

FIG. 1 is a block diagram illustrating a configuration example of a magnetic resonance imaging apparatus according to a first embodiment. As illustrated in FIG. 1, a magnetic resonance imaging apparatus 10 according to the present embodiment includes a static magnetic field magnet 101, a gradient magnetic field coil 103, a gradient magnetic field power supply 104, a bed 105, a bed control circuitry 106, a transmission coil 107, a transmission circuitry 108, a reception coil 109, a reception circuitry 110, a sequence control circuitry 120, and a computer system 130.

Note that the magnetic resonance imaging apparatus 10 does not include a subject P. Furthermore, the configuration of the magnetic resonance imaging apparatus 10 is not limited to the configuration illustrated in FIG. 1. That is, the configuration of the magnetic resonance imaging apparatus 10 is arbitrary. For example, the sequence control circuitry 120 and each unit in the computer system 130 may be integrated or separated as appropriate.

The static magnetic field magnet 101 is a magnet formed in a hollow substantially cylindrical shape, and generates a static magnetic field in an internal space. The static magnetic field magnet 101 is, for example, a superconducting magnet or the like, and is excited by receiving supply of a current from a static magnetic field power supply. The static magnetic field power supply supplies power to the static magnetic field magnet 101. As another example, the static magnetic field magnet 101 may be a permanent magnet, and in this case, the magnetic resonance imaging apparatus 10 may not include a static magnetic field power supply. Furthermore, the static magnetic field power supply may be provided separately from the magnetic resonance imaging apparatus 10.

The gradient magnetic field coil 103 is a coil formed in a hollow cylindrical shape, and is disposed inside the static magnetic field magnet 101. The gradient magnetic field coil 103 is formed by combining three coils corresponding to X, Y, and Z axes orthogonal to each other. It is assumed that a Z-axis direction is the same direction as a direction of the static magnetic field. Furthermore, a Y-axis direction is a vertical direction, and an X-axis direction is a direction perpendicular to the Z-axis and the Y-axis. The gradient magnetic field coil 103 generates a gradient magnetic field to be superimposed on the static magnetic field. Specifically, the three coils in the gradient magnetic field coil 103 are individually supplied with power from the gradient magnetic field power supply 104, and generate a gradient magnetic field whose magnetic field strength changes along each of the X, Y, and Z axes. Furthermore, the gradient magnetic field power supply 104 supplies a current to the gradient magnetic field coil 103 under the control of the sequence control circuitry 120.

The bed 105 includes a top plate 105a on which the subject P is placed. Under the control of the bed control circuitry 106, the bed 105 inserts the top plate 105a into an imaging opening in a state where the subject P is placed on the top plate 105a. Under the control of the computer system 130, the bed control circuitry 106 drives the bed 105 to move the top plate 105a in a longitudinal direction and the vertical direction.

The transmission coil 107 receives the supply of an RF pulse from the transmission circuitry 108, generates a high-frequency magnetic field, and applies the high-frequency magnetic field to the subject P. The transmission circuitry 108 supplies the RF pulse to the transmission coil 107 under the control of the sequence control circuitry 120.

The reception coil 109 is disposed inside the gradient magnetic field coil 103, and receives a magnetic resonance signal (MR signal) emitted from the subject P due to the influence of the high-frequency magnetic field. The reception coil 109 outputs the received magnetic resonance signal to the reception circuitry 110. Note that a configuration in which the reception coil 109 is also used as a transmission coil may be adopted.

The reception circuitry 110 detects the magnetic resonance signal output from the reception coil 109 and generates magnetic resonance data based on the detected magnetic resonance signal. Specifically, the reception circuitry 110 performs analog-digital (AD) conversion on the analog magnetic resonance signal output from the reception coil 109 to generate magnetic resonance data (MR data). Furthermore, the reception circuitry 110 transmits the generated magnetic resonance data to the sequence control circuitry 120. Note that the reception circuitry 110 may be provided on a side of a gantry device including the static magnetic field magnet 101, the gradient magnetic field coil 103, and the like.

The sequence control circuitry 120 performs imaging of the subject P by driving the gradient magnetic field power supply 104, the transmission circuitry 108, and the reception circuitry 110 based on sequence information transmitted from the computer system 130. Here, the sequence information is information defining a procedure for performing the imaging. The sequence information defines the intensity of the current supplied from the gradient magnetic field power supply 104 to the gradient magnetic field coil 103 and the timing of supplying the current, the intensity of the RF pulse supplied from the transmission circuitry 108 to the transmission coil 107 and the timing of applying the RF pulse, the timing at which the reception circuitry 110 detects the magnetic resonance signal, and the like.

The sequence control circuitry 120 is, for example, an integrated circuit such as an application specific integrated circuit (ASIC) or a field programmable gate array (FPGA), or an electronic circuit such as a central processing unit (CPU) or a micro processing unit (MPU).

The computer system 130 performs overall control of the magnetic resonance imaging apparatus 10, generation of a magnetic resonance image, and the like. As illustrated in FIG. 1, the computer system 130 includes a processing circuitry 131, a memory 132, an input device 133, and a display 134.

The processing circuitry 131 is a control circuitry that performs overall control of the magnetic resonance imaging apparatus 10, and is also an arithmetic circuitry that performs various calculations. For example, the processing circuitry 131 according to the present embodiment includes an imaging function 1311, a locator image generation function 1312, a region-of-interest setting function 1313, an output image information setting function 1314, an imaging condition setting function 1315, a reconstructed image generation function 1316, and an output image generation function 1317. The imaging function 1311 corresponds to an imaging unit according to the present embodiment, the locator image generation function 1312 corresponds to a locator image generation unit according to the present embodiment, the region-of-interest setting function 1313 corresponds to a region-of-interest setting unit according to the present embodiment, the output image information setting function 1314 corresponds to an output image information setting unit according to the present embodiment, the imaging condition setting function 1315 corresponds to an imaging condition setting unit according to the present embodiment, the reconstructed image generation function 1316 corresponds to a reconstructed image generation unit according to the present embodiment, and the output image generation function 1317 corresponds to an output image generation unit according to the present embodiment.

In the embodiment of FIG. 1, each processing function performed by the imaging function 1311, the locator image generation function 1312, the region-of-interest setting function 1313, the output image information setting function 1314, the imaging condition setting function 1315, the reconstructed image generation function 1316, and the output image generation function 1317 is stored in the memory 132 in the form of a program executable by a computer. The processing circuitry 131 is a processor that realizes a function corresponding to each of programs by reading and executing the programs from the memory 132. Note that, in FIG. 1, it has been described that the imaging function 1311, the locator image generation function 1312, the region-of-interest setting function 1313, the output image information setting function 1314, the imaging condition setting function 1315, the reconstructed image generation function 1316, and the output image generation function 1317 are implemented by the single processing circuitry 131. However, these functions may be realized by configuring the processing circuitry 131 by combining a plurality of independent processors and executing programs by each processor.

The imaging function 1311 generates sequence information based on the imaging conditions of main imaging set by the imaging condition setting function 1315, and transmits the generated sequence information to the sequence control circuitry 120, thereby causing the sequence control circuitry 120 to perform the main imaging. Furthermore, as a result of the main imaging, the imaging function 1311 arranges the magnetic resonance data transmitted from the sequence control circuitry 120 two-dimensionally or three-dimensionally according to a phase encoding amount and a frequency encoding amount applied by the gradient magnetic field. The magnetic resonance data arranged two-dimensionally or three-dimensionally is called k-space data. Then, the imaging function 1311 stores the k-space data in the memory 132. Note that the main imaging is imaging for collecting images mainly used for diagnosis, and is also referred to as main scanning or the like.

Furthermore, the imaging function 1311 causes the sequence control circuitry 120 to perform locator imaging. For example, the imaging function 1311 causes the sequence control circuitry 120 to perform processing of imaging locator images of three axes including cross sections such as an axial (transverse cross section) image, a sagittal (sagittal cross section) image, and a coronal (coronal cross section) image as locator imaging. Note that the locator imaging is mainly performed prior to the main imaging, and is imaging for collecting a locator image to be referred to when imaging conditions of main imaging are set, and is also referred to as positioning scan or the like. Furthermore, the locator image is also called a scout image.

The locator image generation function 1312 generates a locator image based on magnetic resonance data collected by locator imaging.

The region-of-interest setting function 1313 receives, from an operator, an input operation related to the setting of a region of interest via the input device 133 and sets the region of interest for the locator image according to the received input operation. Furthermore, the region-of-interest setting function 1313 receives, from the operator, an input operation for adjusting the region of interest via the input device 133 and adjusts the region of interest according to the received input operation.

The output image information setting function 1314 sets output image information for generating an output image for the region of interest. The output image information is, for example, information regarding three-dimensional image processing performed on the reconstructed image, information regarding a cross section to be displayed on the display 134, and the like.

Furthermore, the output image is an image generated by applying the region of interest set by the region-of-interest setting function 1313 to the reconstructed image and performing three-dimensional image processing. The three-dimensional image processing is, for example, a maximum intensity projection method, a multi planar reconstruction method, or the like. The maximum intensity projection method is a method of performing projection processing on three-dimensionally constructed data in an arbitrary viewpoint direction and displaying a maximum value in a projection path on a projection surface. Furthermore, the multi planar reconstruction method is a method of constructing cross sections such as Axial (transverse cross section), Sagittal (sagittal cross section), Coronal (coronal cross section), and Radial from three-dimensionally constructed data. The multi planar reconstruction method includes a curved multi planar reconstruction method. That is, examples of the output image include an image displayed using the maximum intensity projection method (hereinafter, a MIP image) and an image displayed using the multi planar reconstruction method (hereinafter, an MPR image). Note that the MPR image includes an image displayed using the curved multi planar reconstruction method (hereinafter, a CPR image).

Furthermore, the output image is not limited to the MIP image or the MPR image. The output image may be, for example, an image displayed using a minimum intensity projection method, an image displayed using a volume rendering method, an image displayed using a surface rendering method, or the like.

The imaging condition setting function 1315 automatically sets the imaging conditions based on the region of interest set by the region-of-interest setting function 1313. Specifically, the imaging condition setting function 1315 automatically sets the imaging conditions such that a FOV of an image imaged by the imaging function 1311 in the main imaging includes the region of interest. Furthermore, the imaging condition setting function 1315 automatically sets the imaging conditions such that a resolution of the image imaged by the imaging function 1311 in the main imaging is higher than a resolution of the locator image.

The imaging conditions are conditions set when imaging such as locator imaging or main imaging is performed. The imaging conditions include, for example, an imaging region including an imaging position and an imaging direction, a field of view (FOV), a matrix size, the number of slices, and the like.

The reconstructed image generation function 1316 generates a reconstructed image by performing reconstruction processing on the k-space data based on the k-space data that is the magnetic resonance data arranged by the imaging function 1311. As the reconstruction processing, for example, fast Fourier transfer (FFT) or the like is used.

The output image generation function 1317 generates an output image based on the reconstructed image generated by the reconstructed image generation function 1316, the region of interest set by the region-of-interest setting function 1313, and the output image information set by the output image information setting function 1314.

The memory 132 includes, for example, a semiconductor memory element such as a random access memory (RAM) or a flash memory, a hard disk, an optical disk, or the like. The memory 132 may include a portable medium such as a universal serial bus (USB) memory and a digital video disk (DVD). The memory 132 stores various processing programs (In addition to the application program, an operating system (OS) and the like are also included.) used in the processing circuitry 131, data necessary for executing the programs, the magnetic resonance data transmitted from the sequence control circuitry 120, the k-space data arranged in the k-space by the imaging function 1311, output images, and the like.

The input device 133 receives, from the operator, various instructions and information inputs. The input device 133 is, for example, a pointing device such as a mouse or a trackball, a selection device such as a mode changeover switch, or an input device such as a keyboard. Furthermore, the input device 133 also includes a touch command screen formed on the display 134 described later.

The display 134 displays various types of information. For example, the display 134 displays a locator image and an output image, and displays a graphical user interface (GUI) for receiving various input operations from the operator. For example, the display 134 is a liquid crystal display (LCD), a cathode ray tube (CRT) display, an organic electro luminescence (EL) display, or the like.

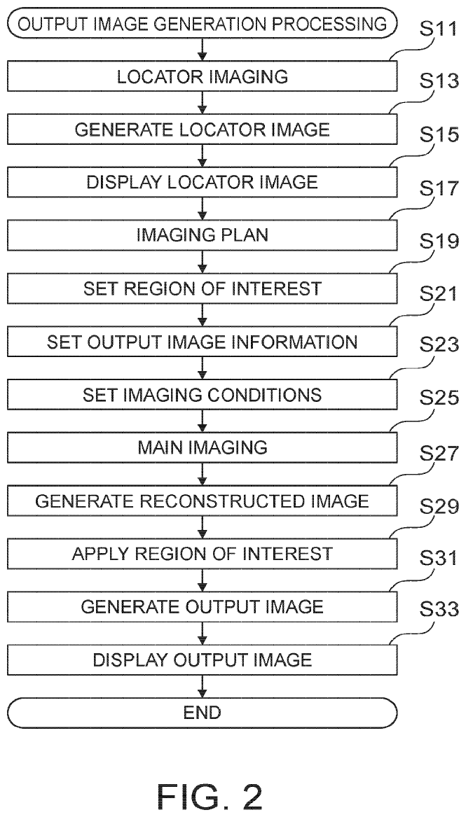

FIG. 2 is a flowchart illustrating the contents of output image generation processing performed by the magnetic resonance imaging apparatus 10 according to the present embodiment. In the output image generation processing illustrated in FIG. 2, the magnetic resonance imaging apparatus 10 sets a region of interest and output image information in a locator image, automatically sets imaging conditions of main imaging based on the region of interest, performs the main imaging based on the imaging conditions, generates a reconstructed image based on magnetic resonance data collected by the main imaging, and generates an output image based on the reconstructed image, the region of interest, and the output image information. For example, this output image generation processing is processing performed in a case where locator imaging is performed.

As illustrated in FIG. 2, first, the magnetic resonance imaging apparatus 10 performs locator imaging (step S11). The processing of performing the locator imaging is realized by the imaging function 1311 in the processing circuitry 131. Specifically, the magnetic resonance imaging apparatus 10 receives, from the operator, an input operation of imaging conditions regarding the locator imaging via the input device 133 and performs the locator imaging.

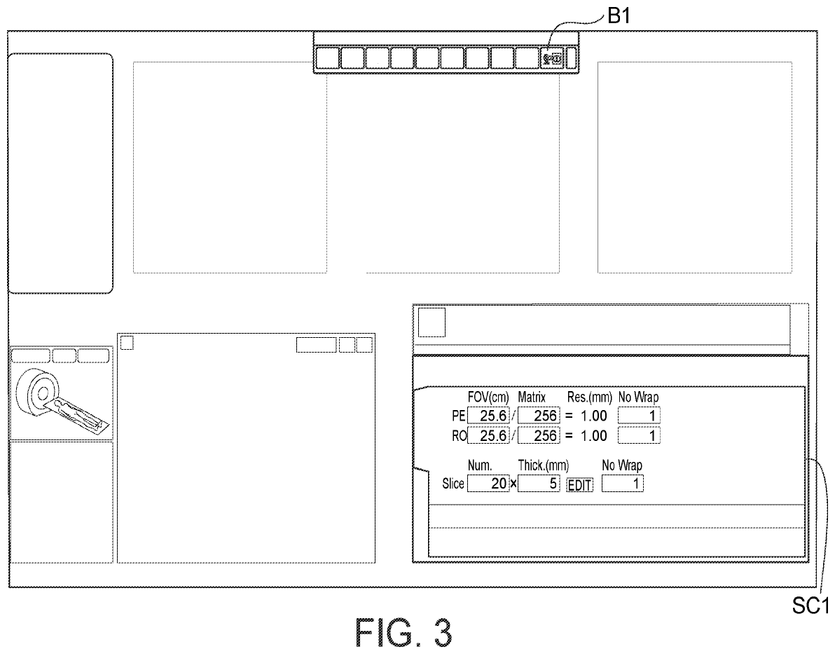

FIG. 3 is a diagram illustrating an example of an imaging condition setting screen for setting imaging conditions to be displayed on the display 134 of the magnetic resonance imaging apparatus 10 according to the first embodiment. As illustrated in FIG. 3, in the present embodiment, on an imaging condition setting screen SC1, as the imaging conditions related to the locator imaging, FOVs in a phase encoding (PE) direction and a read out (RO) direction are set to 25.6 cm and a matrix size is set to 256, and the number of slices is set to 20 and a thickness of one slice is set to 5 mm. The magnetic resonance imaging apparatus 10 performs the locator imaging under imaging conditions of the locator imaging. Note that each value set as the imaging conditions of the locator imaging illustrated in FIG. 3 is an example of the imaging conditions of the locator imaging.

Next, as illustrated in FIG. 2, the magnetic resonance imaging apparatus 10 generates a locator image (step S13). The processing of generating the locator image is realized by the locator image generation function 1312 in the processing circuitry 131. Specifically, the magnetic resonance imaging apparatus 10 performs reconstruction processing on the k-space data based on the k-space data in which the magnetic resonance data collected by the locator imaging in step S11 is arranged, and generates a locator image.

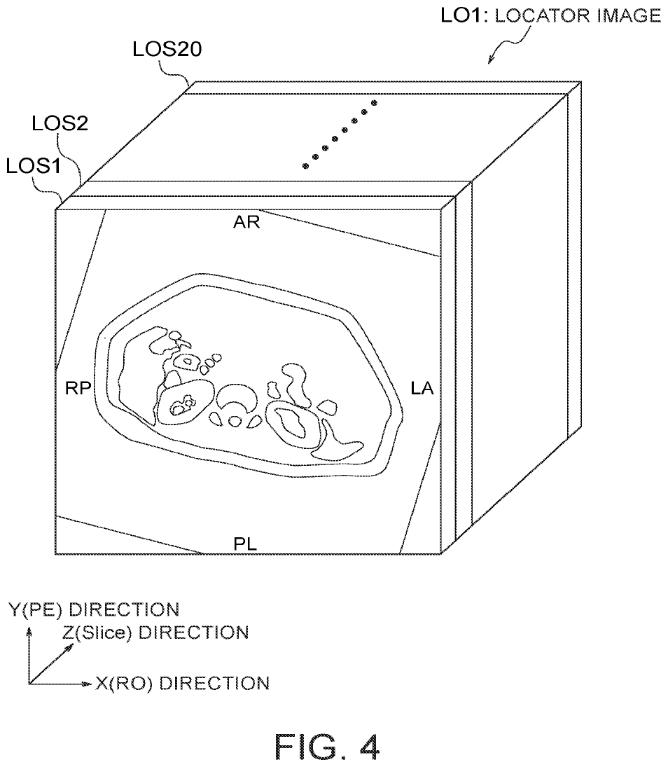

FIG. 4 is a diagram illustrating an example of a locator image generated in the magnetic resonance imaging apparatus 10 according to the present embodiment. As illustrated in FIG. 4, in the present embodiment, a locator image LO1 includes a plurality of substantially parallel slices LOS1 to LOS20 based on the imaging conditions of the locator imaging in step S11. The slices LOS1 to LOS20 constitute a three-dimensional locator image obtained by imaging the abdomen of the subject P. Here, one slice image is an image extending in an X(RO) direction, a Y(PE) direction, and a Z(Slice) direction illustrated in FIG. 4, and the slices LOS1 to LOS20 are arranged in the Z direction to realize a three-dimensional data structure.

Next, as illustrated in FIG. 2, the magnetic resonance imaging apparatus 10 displays the locator image LO1 (step S15). The processing of displaying the locator image LO1 is realized by the region-of-interest setting function 1313 in the processing circuitry 131. Specifically, the magnetic resonance imaging apparatus 10 displays the locator image LO1 on an imaging plan screen by receiving an operation for setting the locator image LO1 on the imaging plan screen from the operator via the input device 133.

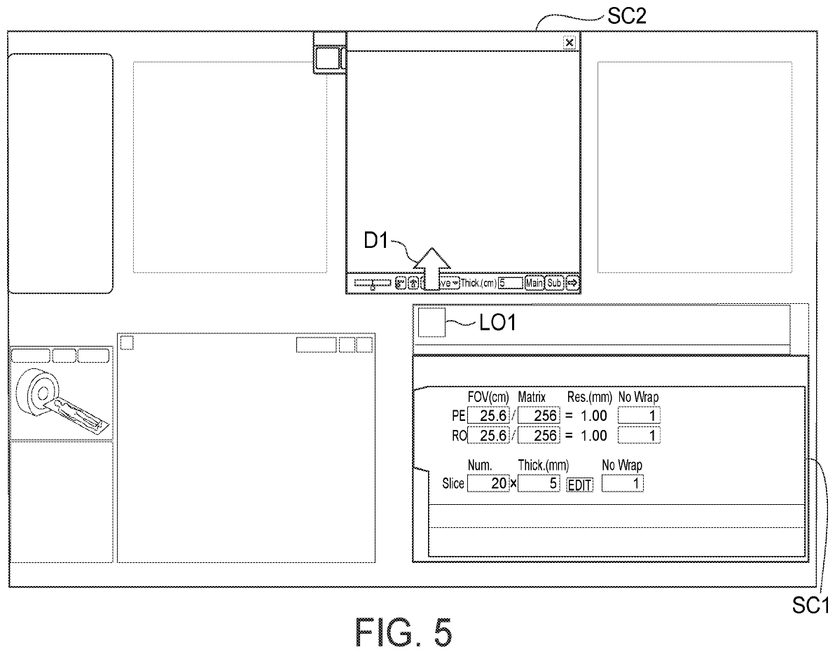

FIG. 5 is a diagram illustrating an example of an imaging plan screen displayed on the display 134 of the magnetic resonance imaging apparatus 10 according to the present embodiment. As illustrated in FIG. 5, the magnetic resonance imaging apparatus 10 opens an imaging plan screen SC2 by receiving selection of a button B1 illustrated in FIG. 3 from the operator via the input device 133. Then, the magnetic resonance imaging apparatus 10 displays the locator image LO1 on the imaging plan screen SC2 by receiving an operation for setting the locator image LO1 on the imaging plan screen SC2 from the operator via the input device 133. The operation for setting the locator image LO1 on the imaging plan screen SC2 is, for example, a drag and drop operation using a mouse as the input device 133. Specifically, as illustrated in FIG. 5, a mouse cursor is moved onto the locator image LO1 displayed as a thumbnail, and a button of the mouse serving as the input device 133 is pressed. Then, while the button of the mouse button is pressed, the mouse cursor is moved in a direction of an arrow D1, and the button of the mouse is released at an arbitrary position on the imaging plan screen SC2. As a result, the locator image LO1 is set on the imaging plan screen SC2.

Note that the operation for setting the locator image LO1 on the imaging plan screen SC2 is not limited to the drag and drop operation using the mouse. That is, the operation for setting the locator image LO1 on the imaging plan screen SC2 is arbitrary, and may be, for example, an operation of clicking the locator image LO1 displayed as a thumbnail.

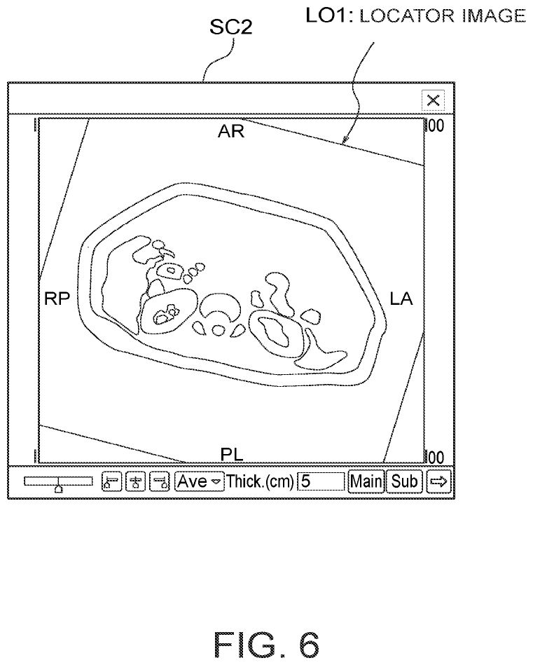

FIG. 6 is a diagram illustrating an example of a locator image LO1 displayed on the imaging plan screen SC2 in the magnetic resonance imaging apparatus 10 according to the first embodiment. In the present embodiment, as illustrated in FIG. 6, one slice image of the locator image LO1 of the abdomen of the subject P collected by imaging of the locator image LO1 is displayed on the imaging plan screen SC2.

Next, as illustrated in FIG. 2, the magnetic resonance imaging apparatus 10 waits until an imaging plan is made by the operator (step S17). That is, the operator performs the imaging plan using the locator image LO1 displayed on the imaging plan screen SC2.

Next, as illustrated in FIG. 2, the magnetic resonance imaging apparatus 10 sets a region of interest (step S19). The processing of setting the region of interest is realized by the region-of-interest setting function 1313 in the processing circuitry 131. Specifically, the magnetic resonance imaging apparatus 10 receives, from the operator, an input operation related to setting of the region of interest, and sets the region of interest for the locator image LO1 according to the input operation. More specifically, in the present embodiment, the magnetic resonance imaging apparatus 10 receives, from the operator, the input operation for superimposing a figure indicating the region of interest on the locator image LO1 via the input device 133, and sets the region of interest.

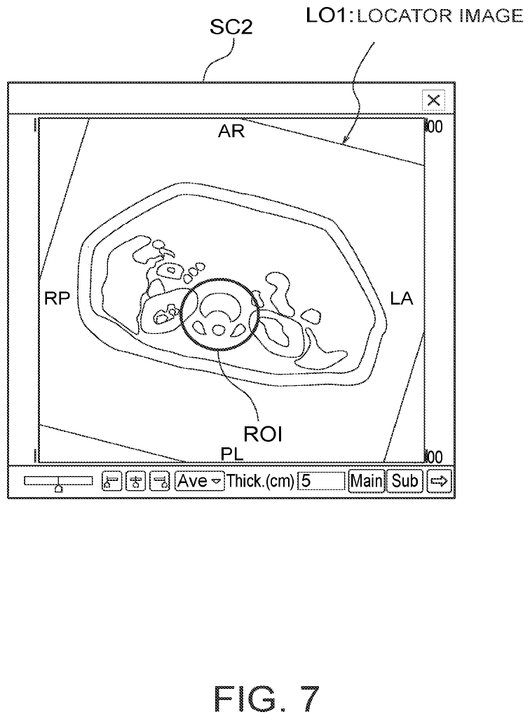

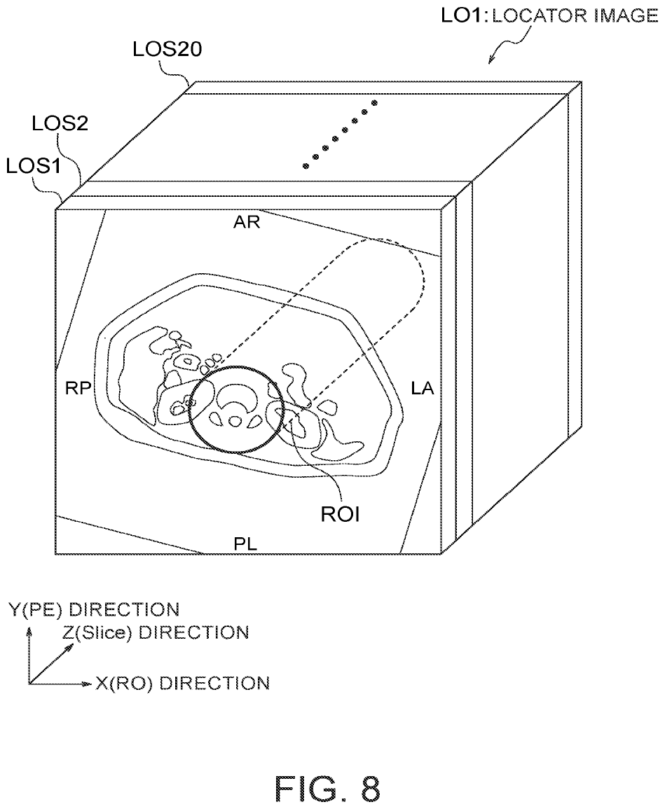

The setting of the region of interest in step S19 will be described in detail with reference to FIGS. 7 and 8. FIG. 7 is a diagram illustrating an example of a figure indicating the region of interest superimposed on the locator image LO1 displayed on the imaging plan screen SC2 by receiving the input operation from the operator in the magnetic resonance imaging apparatus 10 according to the present embodiment. FIG. 8 is a diagram illustrating an example of the region of interest set according to the input operation, from the operator, for superimposing a figure indicating the region of interest on the locator image LO1 in the magnetic resonance imaging apparatus 10 according to the present embodiment. As illustrated in FIG. 7, in the present embodiment, the magnetic resonance imaging apparatus 10 receives, from the operator, the input operation for superimposing a circle, which is a figure indicating the region of interest ROI, on the slice of the locator image LO1 displayed on the imaging plan screen SC2 via the input device 133. When the input operation for superimposing the figure on the slice of the locator image LO1 is received from the operator, the magnetic resonance imaging apparatus 10 also superimposes a circle, which is a figure indicating the region of interest ROI, on another slice of the locator image LO1. That is, as illustrated in FIG. 8, the magnetic resonance imaging apparatus 10 sets a cylindrical region of interest ROI for the locator image LO1.

Note that, in step S19, the shape of the figure to be superimposed on the locator image LO1 is a circle, but the shape of the figure to be superimposed on the locator image LO1 is not limited thereto. That is, the shape of the figure to be superimposed on the locator image LO1 is arbitrary, and may be, for example, a polygon, a rectangle, an ellipse, or the like.

Furthermore, in step S19, the magnetic resonance imaging apparatus 10 can receive, from the operator, an input operation for adjusting a figure indicating the region of interest ROI superimposed on the locator image LO1 via the input device 133, and adjust the region of interest ROI according to the input operation. That is, the magnetic resonance imaging apparatus 10 may receive, from the operator, the input operation for adjusting a position and/or a size of a figure indicating the region of interest ROI superimposed on the locator image LO1 as the input operation for adjusting the region of interest ROI for each slice of the locator image LO1, and adjust the region of interest ROI of the locator image LO1 according to the input operation.

Next, as illustrated in FIG. 2, the magnetic resonance imaging apparatus 10 sets output image information (step S21). The processing of setting the output image information is realized by the output image information setting function 1314 in the processing circuitry 131. Specifically, the magnetic resonance imaging apparatus 10 displays an output image information setting screen on the display 134 and set the output image information by causing the user to select output image information.

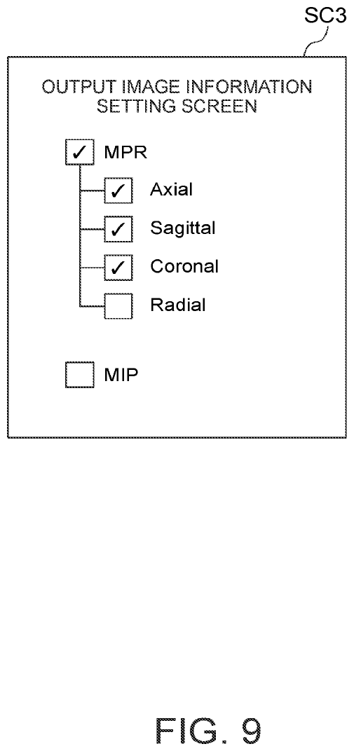

FIG. 9 is a diagram illustrating an example of an output image information setting screen displayed on the display 134 of the magnetic resonance imaging apparatus 10 according to the present embodiment. As illustrated in FIG. 9, MPR or MIP is selectively displayed on an output image information setting screen SC3 as three-dimensional image processing performed on the reconstructed image when the output image is generated. Furthermore, in the MPR, cross sections such as Axial (transverse cross section), Sagittal (sagittal cross section), Coronal (coronal cross section), and Radial are selectively displayed. In the present embodiment, as illustrated in FIG. 9, the operator selects MPR as three-dimensional image processing, and selects cross sections of Axial, Sagittal, and Coronal as cross sections to be displayed on the display 134 in MPR. Therefore, the magnetic resonance imaging apparatus 10 sets MPR and cross sections of Axial, Sagittal, and Coronal as output image information.

Note that, in the example illustrated in FIG. 9, the magnetic resonance imaging apparatus 10 sets the cross sections of Axial, Sagittal, and Coronal as the output image information, but the number of cross sections set as the output image information is not limited to three. That is, the number of cross sections set as the output image information is arbitrary according to the selection of the operator, and for example, the magnetic resonance imaging apparatus 10 may set one or two cross sections among Axial, Sagittal, Coronal, and Radial as the output image information or may set all the cross sections of Axial, Sagittal, Coronal, and Radial as the output image information according to the selection of the operator.

Next, as illustrated in FIG. 2, the magnetic resonance imaging apparatus 10 sets imaging conditions (step S23). The processing of setting the imaging conditions is realized by the imaging condition setting function 1315 in the processing circuitry 131. Specifically, the magnetic resonance imaging apparatus 10 automatically sets the imaging conditions for the main imaging based on the region of interest ROI set for the locator image LO1 in step S19.

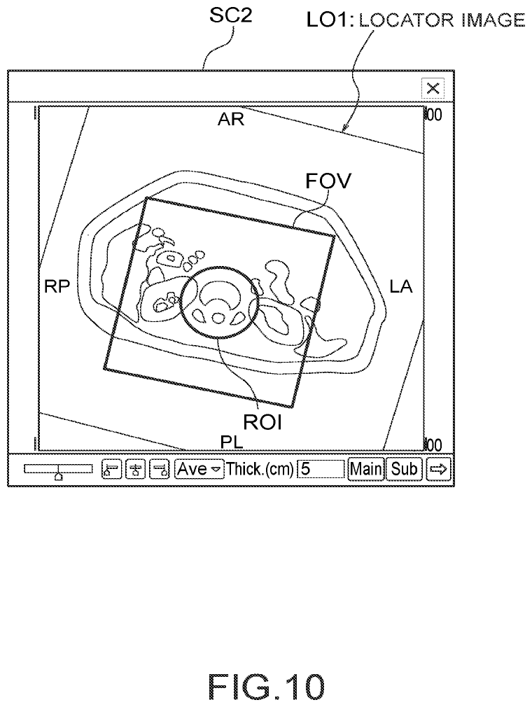

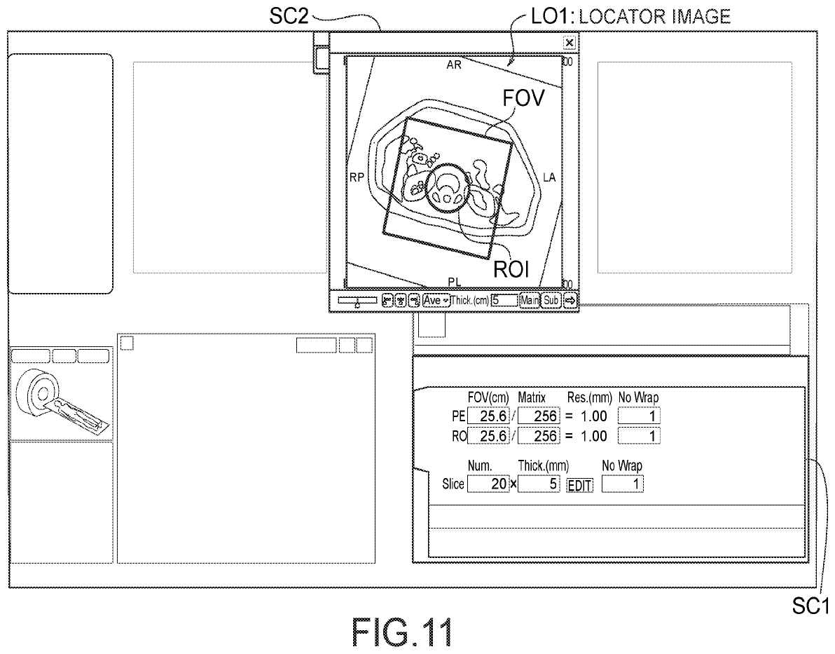

The setting of the imaging conditions of the main imaging in step S23 will be described in detail with reference to FIGS. 10 and 11. FIG. 10 is a diagram illustrating an example of a case where a FOV of an image in the main imaging is set in the magnetic resonance imaging apparatus 10 according to the present embodiment. FIG. 11 is a diagram illustrating an example of an imaging condition of the main imaging set based on the region of interest ROI set for the locator image LO1 in the magnetic resonance imaging apparatus 10 according to the present embodiment. A rectangular figure illustrated in FIG. 10 indicates the FOV.

As illustrated in FIG. 10, the magnetic resonance imaging apparatus 10 automatically sets a size of the FOV that is one of the imaging conditions such that the FOV of the imaged image indicated by the rectangular figure includes the region of interest ROI. Therefore, in the present embodiment, as illustrated in FIG. 11, the magnetic resonance imaging apparatus 10 sets, to 12.8 cm, the size of any of the FOVs in the PE direction and the RO direction of the image imaged in the main imaging.

Furthermore, the magnetic resonance imaging apparatus 10 automatically sets a matrix size, which is one of the imaging conditions, such that a resolution of the image is higher than a resolution of the locator image LO1. In the present embodiment, in a case where a FOV of the image imaged in the main imaging is smaller than a FOV of the locator image LO1, the magnetic resonance imaging apparatus 10 sets a matrix size of the image imaged in the main imaging to be the same as a matrix size of the locator image LO1, thereby automatically setting the matrix size, which is one of the imaging conditions, such that a resolution of the image imaged by the imaging function 1311 in the main imaging becomes higher than a resolution of the locator image LO1.

Specifically, in the present embodiment, as illustrated in FIG. 3, the FOVs in both the PE direction and the RO direction of the locator image LO1 are 25.6 cm, and as illustrated in FIG. 11, the FOVs in both the PE direction and the RO direction of the image imaged in the main imaging are 12.8 cm. That is, since the FOV of the image imaged in the main imaging is smaller than the FOV of the locator image LO1, the matrix size, which is one of the imaging conditions, is automatically set such that both the matrix size of the image imaged in the main imaging and the matrix size of the locator image LO1 are 256. As a result, the resolution of the image imaged in the main imaging can be made higher than the resolution of the locator image LO1.

Moreover, the number of slices set in the main imaging may be the same as the number of slices set in the locator imaging, or may be larger than the number of slices set in the locator imaging. In the present embodiment, the magnetic resonance imaging apparatus 10 sets the number of slices such that the number of slices set in the main imaging is the same as the number of slices set in the locator imaging.

Furthermore, a slice thickness set in the main imaging may be the same as a slice thickness set in the locator imaging, or may be thinner than a slice thickness set in the locator imaging. In the present embodiment, the magnetic resonance imaging apparatus 10 sets the slice thickness so that the slice thickness set in the main imaging and the slice thickness set in the locator imaging become the same.

From the above, in the present embodiment, by the magnetic resonance imaging apparatus 10 automatically setting the imaging conditions for the main imaging, as illustrated in FIG. 11, in the imaging condition setting screen SC1, as the imaging conditions for the main imaging, FOVs in the phase encoding (PE) direction and the read out (RO) direction are set to 12.8 cm, the matrix size is set to 256, the number of slices is set to 20, and the thickness of one slice is set to 5 mm. Note that each value set as the imaging conditions of the main imaging illustrated in FIG. 11 is an example of the imaging conditions of the main imaging.

Next, as illustrated in FIG. 2, the magnetic resonance imaging apparatus 10 performs the main imaging (step S25). The processing of performing the main imaging is realized by the imaging function 1311 in the processing circuitry 131. Specifically, the magnetic resonance imaging apparatus 10 generates sequence information based on the imaging conditions set in step S23 and transmits the generated sequence information to the sequence control circuitry 120, and the sequence control circuitry 120 drives the gradient magnetic field power supply 104, the transmission circuitry 108, and the reception circuitry 110 to perform the main imaging of the subject P.

Furthermore, the magnetic resonance imaging apparatus 10 arranges the magnetic resonance data transmitted from the sequence control circuitry 120 two-dimensionally or three-dimensionally according to the phase encoding amount or the frequency encoding amount applied by the gradient magnetic field as a result of the main imaging by the imaging function 1311. Then, k space data which is the magnetic resonance data arranged two-dimensionally or three-dimensionally is stored in the memory 132.

Next, as illustrated in FIG. 2, the magnetic resonance imaging apparatus 10 generates a reconstructed image (step S27). The processing of generating the reconstructed image is realized by the reconstructed image generation function 1316 in the processing circuitry 131. Specifically, the magnetic resonance imaging apparatus 10 generates a reconstructed image based on the magnetic resonance data collected by the main imaging performed by the imaging function 1311. More specifically, the magnetic resonance imaging apparatus 10 performs reconstruction processing on the k-space data that is the arranged magnetic resonance data to generate a reconstructed image. For example, fast Fourier transfer (FFT) or the like is used for the reconstruction processing.

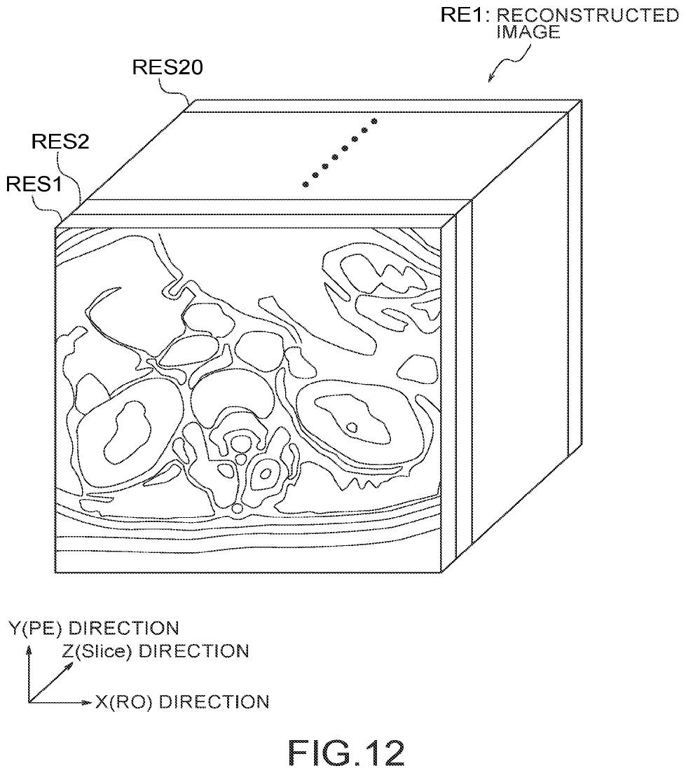

FIG. 12 is a diagram illustrating an example of a reconstructed image generated by the reconstruction processing in the magnetic resonance imaging apparatus 10 according to the present embodiment. As illustrated in FIG. 12, in the present embodiment, a reconstructed image RE1 includes a plurality of substantially parallel slices RES1 to RES20 based on the imaging conditions of the main imaging set in step S23. The slices RES1 to RES20 constitute a three-dimensional reconstructed image RE1 including the region of interest ROI of the subject P. Here, one slice image is an image extending in the X(RO) direction, the Y (PE)direction, and the Z(Slice) direction illustrated in FIG. 12, and the slices RES1 to RES20 are arranged in the Z direction to realize a three-dimensional data structure.

Next, as illustrated in FIG. 2, the magnetic resonance imaging apparatus 10 applies the region of interest ROI to the reconstructed image RE1 (step S29). The processing of applying the region of interest ROI is realized by the output image generation function 1317 in the processing circuitry 131. Specifically, the magnetic resonance imaging apparatus 10 applies the region of interest ROI set by the region-of-interest setting function 1313 in step S19 to the reconstructed image RE1 generated in step S27.

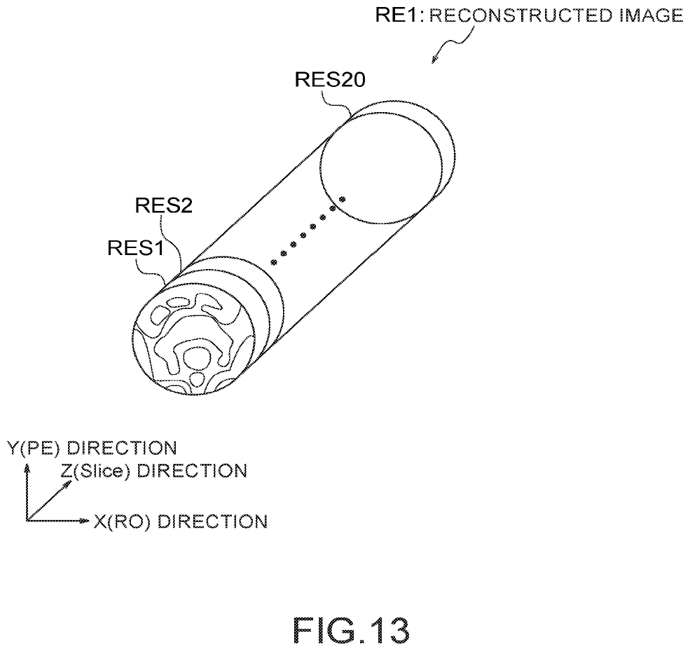

FIG. 13 is a diagram illustrating an example of the reconstructed image RE1 to which the region of interest ROI is applied in the magnetic resonance imaging apparatus 10 according to the present embodiment. As illustrated in FIG. 13, in the present embodiment, the magnetic resonance imaging apparatus 10 applies the region of interest ROI to the reconstructed image RE1 so as to cut out a cylindrical region of interest ROI set in step S19 from the reconstructed image RE1. Note that, in the present embodiment, the region of interest ROI is applied to the reconstructed image RE1 by cutting out the region of interest ROI from the reconstructed image RE1, but the method of applying the region of interest ROI to the reconstructed image RE1 is not limited thereto. That is, a method of applying the region of interest ROI to the reconstructed image RE1 is arbitrary, and for example, a pixel value other than the region of interest ROI to the reconstructed image RE1 may be set to 0.

Next, as illustrated in FIG. 2, the magnetic resonance imaging apparatus 10 generates an output image (step S31). The processing of generating the output image is realized by the output image generation function 1317 in the processing circuitry 131. Specifically, the magnetic resonance imaging apparatus 10 generates an output image based on the reconstructed image RE1 generated in step S27, the region of interest ROI set in step S19, and the output image information set in step S21.

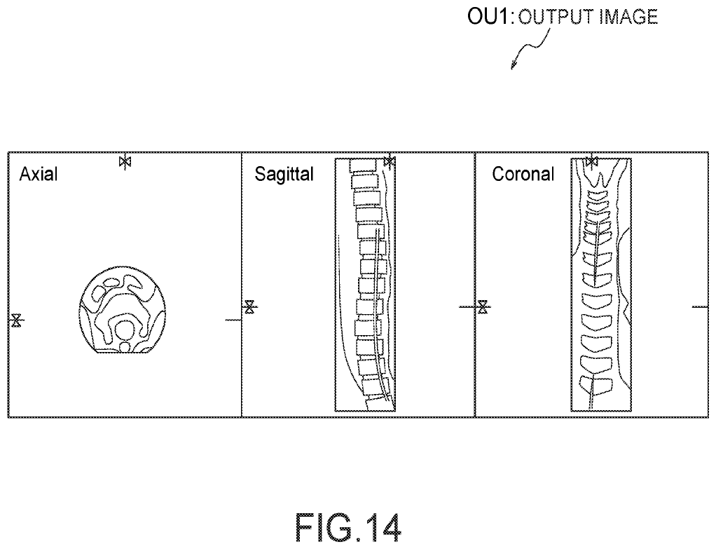

FIG. 14 is a diagram illustrating an example of an output image to be generated. As illustrated in FIG. 14, in the present embodiment, the magnetic resonance imaging apparatus 10 performs the MPR set in step S21 on the reconstructed image RE1 cut out in the region of interest ROI in step S27 to generate images of the respective cross sections of Axial, Sagittal, and Coronal, thereby generating an MPR image regarding the spine as an output image OU1.

Next, as illustrated in FIG. 2, the magnetic resonance imaging apparatus 10 displays the output image OU1 (step S33). The processing of displaying the output image OU1 is realized by the output image generation function 1317 in the processing circuitry 131. Specifically, the magnetic resonance imaging apparatus 10 displays the output image OU1 generated in step S31 on the display 134. In the present embodiment, the magnetic resonance imaging apparatus 10 displays an image of each of cross sections of Axial, Sagittal, and Coronal on the display 134 as the MPR image generated in step S31.

In step S33, the output image OU1 is displayed on the display 134, whereby the output image generation processing according to the present embodiment ends.

As described above, according to the magnetic resonance imaging apparatus 10 of the present embodiment, the magnetic resonance imaging apparatus 10 receives, from the operator, the input operation related to the setting of the region of interest ROI with respect to the locator image LO1, sets the region of interest ROI according to the received input operation, automatically sets the imaging conditions based on the region of interest ROI, and performs imaging based on the imaging conditions. Therefore, it is possible to shorten the time required for setting the imaging conditions while reducing the burden on the operator. That is, in the present embodiment, the magnetic resonance imaging apparatus 10 receives, from the operator, the input operation for superimposing a figure indicating the region of interest ROI on the locator image LO1, sets the region of interest ROI, automatically sets the size of the FOV, which is one of the imaging conditions, based on the region of interest ROI such that the FOV of the image to be imaged includes the region of interest ROI indicated by the figure, automatically sets the matrix size, which is one of the imaging conditions, based on the region of interest ROI such that the resolution of the image is higher than the resolution of the locator image LO1, and performs the main imaging based on the size of the FOV and the matrix size. Therefore, the operator can set the imaging conditions without manually setting the imaging conditions according to the region of interest ROI while referring to the locator image LO1.

Moreover, since the magnetic resonance imaging apparatus 10 sets the output image information for generating an output image for the region of interest ROI, generates the reconstructed image RE1 based on the magnetic resonance data collected by the main imaging, and generates the output image OU1 based on the output image information, the reconstructed image RE1, and the region of interest ROI, it is possible to shorten the time required for generating the output image OU1 while reducing the burden on the operator. That is, in the present embodiment, the magnetic resonance imaging apparatus 10 sets the three-dimensional image processing to be performed on the reconstructed image and the cross section to be displayed on the display 134 as the output image information, cuts out the region of interest ROI from the reconstructed image RE1, performs the three-dimensional image processing on the reconstructed image RE1 cut out in the region of interest ROI, generates the image of each cross section set in the reconstructed image RE1 on which the three-dimensional image processing has been performed, and generates the output image. Therefore, the operator can generate the output image without creating an image to which the region of interest is manually applied, and manually performing the three-dimensional image processing on the created image.

### Second Embodiment

In the first embodiment described above, the magnetic resonance imaging apparatus 10 receives, from the operator, an input operation for superimposing a figure indicating the region of interest ROI on the locator image, sets the region of interest ROI, and generates the MPR image as the output image. However, the present invention is not limited to this. In the second embodiment, a magnetic resonance imaging apparatus 10 that receives, from an operator, an input operation for setting a pixel value in a locator image, sets a region of interest ROI, and generates a MIP image as an output image will be described. Hereinafter, portions different from the above-described first embodiment will be described. Note that the configuration of the magnetic resonance imaging apparatus 10 according to the present embodiment is the same as that in FIG. 1, and thus description thereof is omitted.

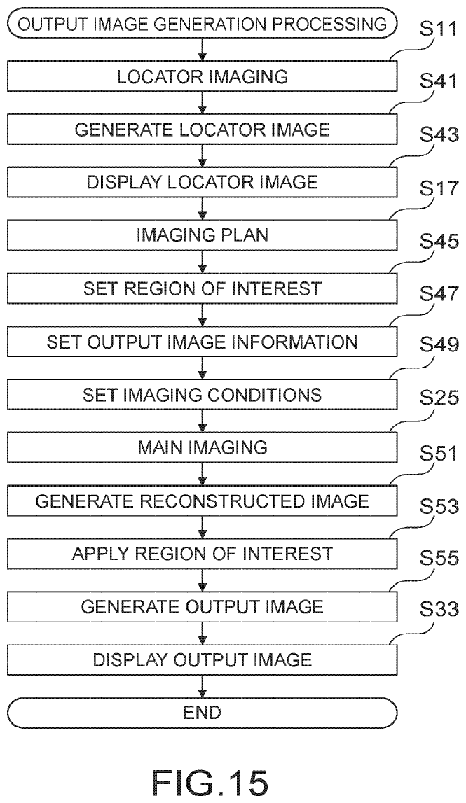

FIG. 15 is a flowchart illustrating the contents of output image generation processing performed by the magnetic resonance imaging apparatus 10 according to the present embodiment, and is a diagram corresponding to FIG. 2 in the above-described first embodiment. In this output image generation processing, the magnetic resonance imaging apparatus 10 sets a region of interest and output image information in a locator image, automatically sets imaging conditions for main imaging based on the region of interest, performs the main imaging based on the imaging conditions, generates a reconstructed image based on magnetic resonance data collected by the main imaging, and generates an output image based on the reconstructed image, the region of interest, and the output image information. For example, this output image generation processing is processing performed in a case where locator imaging is performed. Note that the processing in step S11 is the same as that in FIG. 2 in the first embodiment described above, and thus description thereof is omitted.

Next, as illustrated in FIG. 15, the magnetic resonance imaging apparatus 10 generates a locator image (step S41). The processing of generating the locator image is realized by the locator image generation function 1312 in the processing circuitry 131. Specifically, the magnetic resonance imaging apparatus 10 performs reconstruction processing on the k-space data based on the k-space data in which the magnetic resonance data collected by the locator imaging in step S11 is arranged, and generates a locator image.

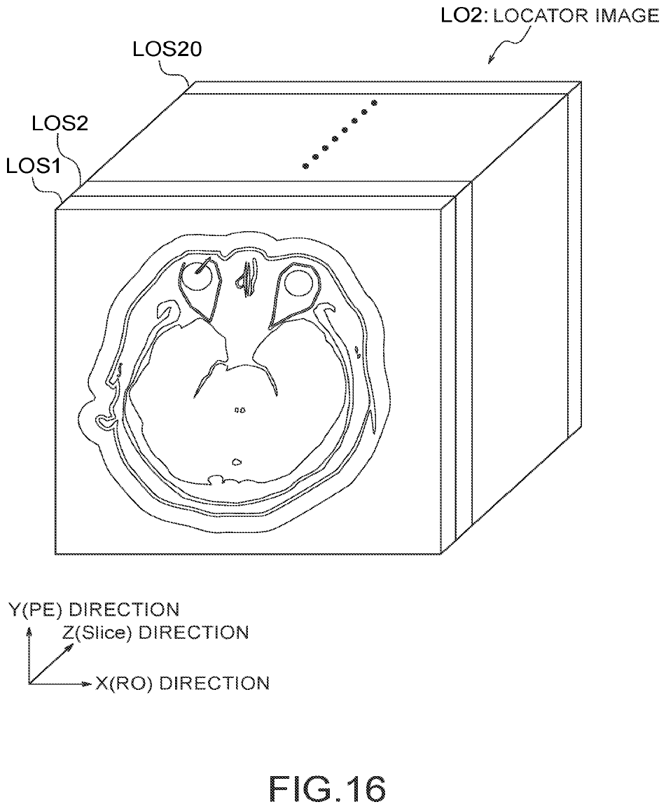

FIG. 16 is a diagram illustrating an example of the locator image generated by the magnetic resonance imaging apparatus 10 according to the present embodiment, and is a diagram corresponding to FIG. 4 in the above-described first embodiment. As illustrated in FIG. 16, in the present embodiment, a locator image LO2 includes a plurality of substantially parallel slices LOS1 to LOS20 based on the imaging conditions of the locator imaging set in step S11. The slices LOS1 to LOS20 constitute a three-dimensional locator image obtained by imaging the head of the subject P. Here, one slice image is an image extending in the X(RO) direction, the Y(PE) direction, and the Z(Slice) direction illustrated in FIG. 16, and the slices LOS1 to LOS20 are arranged in the Z direction to realize a three-dimensional data structure.

Next, as illustrated in FIG. 15, the magnetic resonance imaging apparatus 10 displays the locator image LO2 (step S43). The processing of displaying the locator image LO2 is realized by the region-of-interest setting function 1313 in the processing circuitry 131. Specifically, the magnetic resonance imaging apparatus 10 displays the locator image LO2 on an imaging plan screen SC2 by receiving, from the operator, an operation for setting the locator image on the imaging plan screen SC2 via the input device 133. Note that the operation for setting the locator image on the imaging plan screen SC2 by the operator is the same as the description in the first embodiment described above, and thus the description thereof will be omitted.

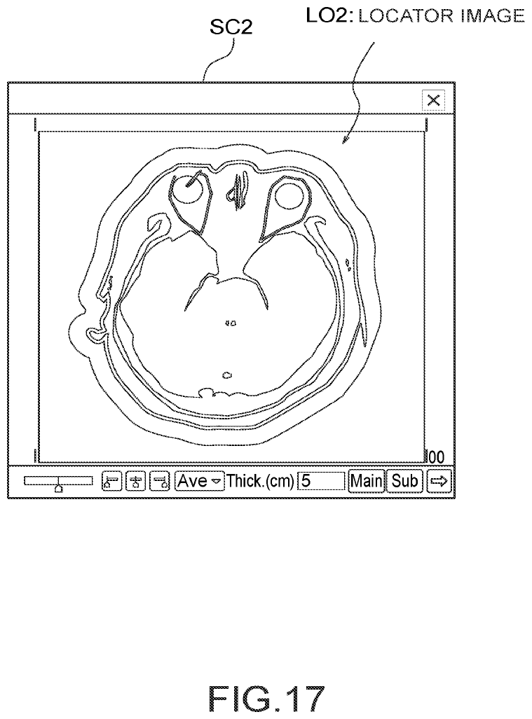

FIG. 17 is a diagram illustrating an example of the locator image LO2 displayed on the imaging plan screen SC2, and is a diagram corresponding to FIG. 6 in the first embodiment described above. In the present embodiment, as illustrated in FIG. 17, one slice image of the locator image LO2 of the head of the subject P collected by the locator imaging is displayed on the imaging plan screen SC2. Note that the processing in step S17 illustrated in FIG. 15 is equivalent to that in FIG. 2 in the first embodiment described above, and thus description thereof is omitted.

Next, as illustrated in FIG. 15, the magnetic resonance imaging apparatus 10 sets the region of interest ROI (step S45). The processing of setting the region of interest ROI is realized by the region-of-interest setting function 1313 in the processing circuitry 131. Specifically, the magnetic resonance imaging apparatus 10 receives, from the operator, the input operation related to setting of the region of interest ROI, and sets the region of interest ROI in the locator image LO2 according to the received input operation. More specifically, in the present embodiment, the magnetic resonance imaging apparatus 10 receives, from the operator, the input operation for setting a pixel value in the locator image LO2 via the input device 133, and sets the region of interest ROI.

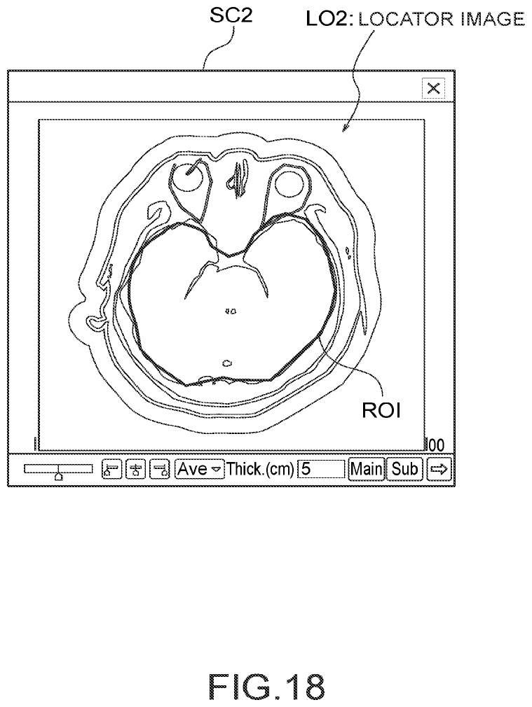

The setting of the region of interest ROI in step S45 will be described in detail with reference to FIG. 18. FIG. 18 is a diagram illustrating an example of a region of interest ROI set by receiving the input operation for setting a pixel value with respect to the locator image LO2 displayed on the imaging plan screen SC2 in the magnetic resonance imaging apparatus 10 according to the present embodiment. In step S45, the operator performs the input operation for setting a pixel value on the slice image of the locator image LO2 displayed on the imaging plan screen SC2 via the input device 133. Upon receiving the input operation for setting the pixel value, the magnetic resonance imaging apparatus 10 performs segmentation processing by threshold processing using the pixel value on the slice image of the locator image LO2 and other slice images of the locator image LO2 displayed on the imaging plan screen SC2 based on the pixel value set by receiving the input operation for setting the pixel value, and sets the region of interest ROI. In the present embodiment, as illustrated in FIG. 18, the region of interest ROI is set so as to surround the region of the brain imaged in the locator image LO2 of the head by the segmentation processing.

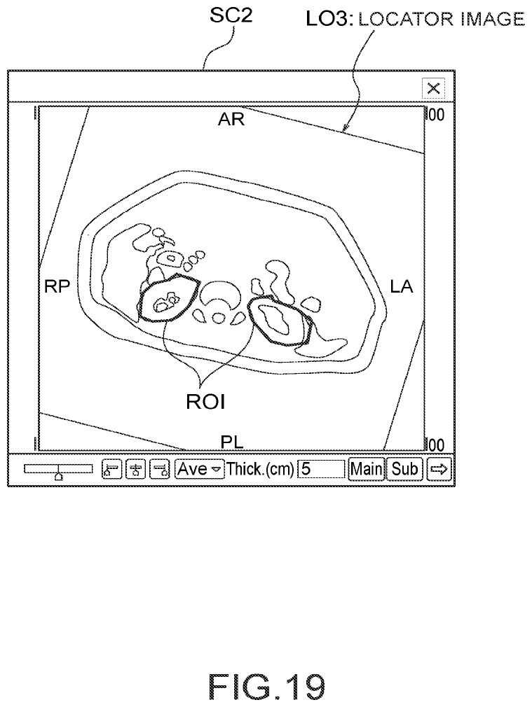

In the example illustrated in FIG. 18, the magnetic resonance imaging apparatus 10 sets one region of interest ROI for one locator image LO2, but the present invention is not limited thereto. That is, a plurality of regions of interest ROI may be set for one locator image. FIG. 19 is a diagram illustrating an example of a plurality of regions of interest ROIs set for one locator image in the magnetic resonance imaging apparatus according to the present embodiment. A locator image LO3 illustrated in FIG. 19 is a locator image of the abdomen. The operator performs the input operation for setting a pixel value on the slice image of the locator image LO3 displayed on the imaging plan screen SC2 via the input device 133. Upon receiving the input operation for setting the pixel value, the magnetic resonance imaging apparatus 10 performs segmentation processing by threshold processing using the pixel value on the slice image of the locator image LO3 and the other slice image of the locator image LO3 displayed on the imaging plan screen SC2 based on the pixel value set by receiving the input operation for setting the pixel value, and sets two regions of interest ROIs. As described above, the magnetic resonance imaging apparatus 10 may set the plurality of regions of interest ROIs by segmentation processing for one locator image.

Furthermore, in step S45, the magnetic resonance imaging apparatus 10 can receive, from the operator via the input device 133, an input operation for adjusting a pixel value with respect to the locator image LO2 displayed on the imaging plan screen SC2, and adjust the region of interest ROI according to the received input operation. That is, the magnetic resonance imaging apparatus 10 may receive, from the operator, the input operation for adjusting the contrast or the like of the locator image LO2 as the input operation for adjusting the region of interest ROI, and adjust the region of interest ROI of the locator image LO2 according to the received input operation.

Next, as illustrated in FIG. 15, the magnetic resonance imaging apparatus 10 sets output image information (step S47). The processing of setting the output image information is realized by the output image information setting function 1314 in the processing circuitry 131. Specifically, the magnetic resonance imaging apparatus 10 displays an output image information setting screen on the display 134 and set the output image information by causing the user to select output image information.

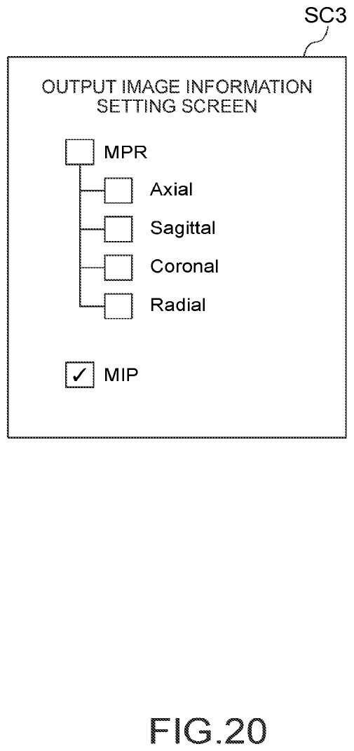

FIG. 20 is a diagram illustrating an example of an output image information setting screen SC3 displayed on the display 134 in the magnetic resonance imaging apparatus 10 according to the present embodiment. As illustrated in FIG. 20, MPR or MIP is selectively displayed on the output image information setting screen SC3 as the three-dimensional image processing performed on the reconstructed image when the output image is generated. Furthermore, in the MPR, cross sections such as Axial, Sagittal, Coronal, and Radial are selectively displayed. In the present embodiment, since the operator selects MIP as the three-dimensional image processing, the magnetic resonance imaging apparatus sets the MIP as the output image information.

Next, as illustrated in FIG. 15, the magnetic resonance imaging apparatus 10 sets imaging conditions (step S49). The processing of setting the imaging conditions is realized by the imaging condition setting function 1315 in the processing circuitry 131. Specifically, the magnetic resonance imaging apparatus 10 automatically sets the imaging conditions based on the region of interest ROI set for the locator image LO2 in step S45.

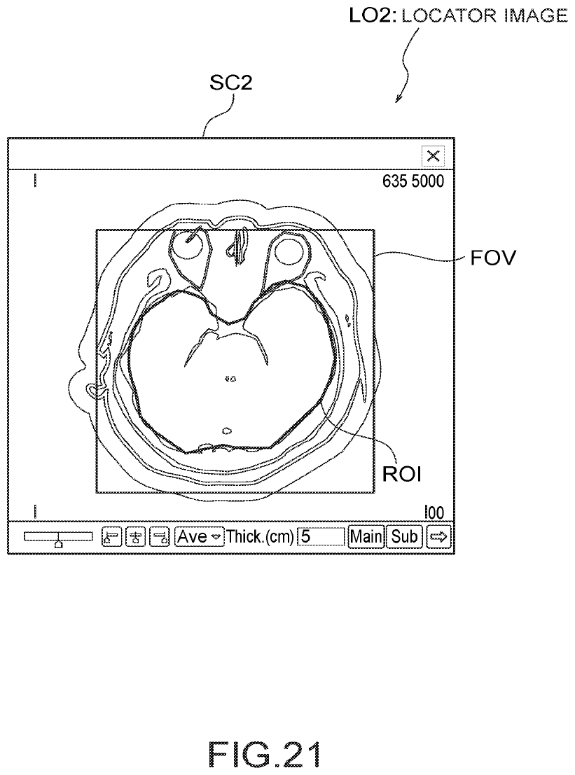

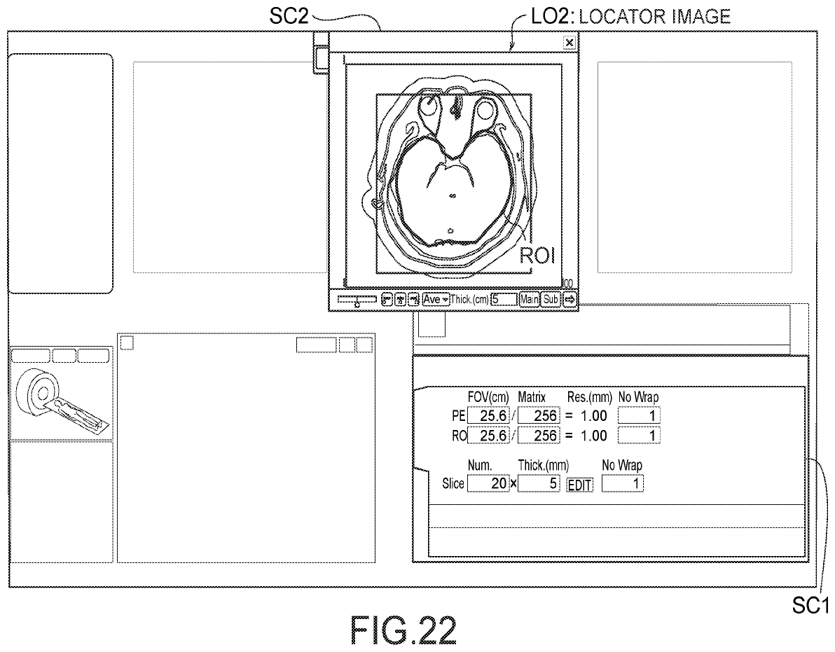

The setting of the imaging condition in step S49 will be described in detail with reference to FIGS. 21 and 22. FIG. 21 is a diagram illustrating an example of a case where a FOV of an image in the main imaging is set in the magnetic resonance imaging apparatus 10 according to the present embodiment, and is a diagram corresponding to FIG. 10 in the first embodiment described above. FIG. 22 is a diagram illustrating an example of imaging conditions set based on the region of interest ROI set for the locator image LO2 in the magnetic resonance imaging apparatus 10 according to the present embodiment, and is a diagram corresponding to FIG. 11 in the above-described first embodiment. A rectangular figure illustrated in FIG. 21 indicates the FOV.

As illustrated in FIG. 21, the magnetic resonance imaging apparatus automatically sets a size of the FOV that is one of the imaging conditions such that the FOV of the imaged image indicated by the rectangular figure includes the region of interest ROI. Therefore, in the present embodiment, as illustrated in FIG. 22, the magnetic resonance imaging apparatus 10 sets, to 19.2 cm, the size of any of the FOVs in the PE direction and the RO direction of the image imaged in the main imaging.

Furthermore, the magnetic resonance imaging apparatus 10 automatically sets a matrix size, which is one of the imaging conditions, such that a resolution of the image is higher than a resolution of the locator image LO2. In the present embodiment, in a case where a FOV of the image imaged in the main imaging is smaller than a FOV of the locator image LO2, the magnetic resonance imaging apparatus 10 sets a matrix size of the image imaged in the main imaging to be the same as a matrix size of the locator image LO2, thereby automatically setting the matrix size, which is one of the imaging conditions, such that a resolution of the image imaged by the imaging function 1311 in the main imaging becomes higher than a resolution of the locator image LO2.

Specifically, in the present embodiment, as illustrated in FIG. 3, the FOVs in both the PE direction and the RO direction of the locator image LO2 are 25.6 cm, and as illustrated in FIG. 22, the FOVs in both the PE direction and the RO direction of the image imaged in the main imaging are 19.2 cm. That is, since the FOV of the image imaged in the main imaging is smaller than the FOV of the locator image LO2, the matrix size, which is one of the imaging conditions, is automatically set such that both the matrix size of the image imaged in the main imaging and the matrix size of the locator image LO2 are 256. As a result, the resolution of the image imaged in the main imaging can be made higher than the resolution of the locator image LO2.

Note that the number of slices and the slice thickness set in the main imaging are the same as the description of the number of slices and the slice thickness in the first embodiment described above, and thus the description thereof will be omitted.

From the above, in the present embodiment, by the magnetic resonance imaging apparatus 10 automatically setting the imaging conditions for main imaging, the FOVs in the phase encoding (PE) direction and the read out (RO) direction are set to 19.2 cm and the matrix size is set to 256, and the number of slices is set to 20 and the thickness of one slice is set to 5 mm as the imaging conditions for main imaging on the imaging condition setting screen SC1 illustrated in FIG. 22. Note that, each value set as the imaging conditions of the main imaging illustrated in FIG. 22 is an example of the imaging conditions of the main imaging. Note that the processing in step S25 illustrated in FIG. 15 is equivalent to that in FIG. 2 in the first embodiment described above, and thus description thereof is omitted.

Next, as illustrated in FIG. 15, the magnetic resonance imaging apparatus 10 generates a reconstructed image (step S51). The processing of generating the reconstructed image is realized by the reconstructed image generation function 1316 in the processing circuitry 131. Specifically, the magnetic resonance imaging apparatus 10 generates a reconstructed image based on the magnetic resonance data collected by the main imaging performed by the imaging function 1311. More specifically, the magnetic resonance imaging apparatus 10 performs reconstruction processing on the k-space data that is the magnetic resonance data arranged two-dimensionally or three-dimensionally to generate a reconstructed image. For example, fast Fourier transfer (FFT) or the like is used for the reconstruction processing.

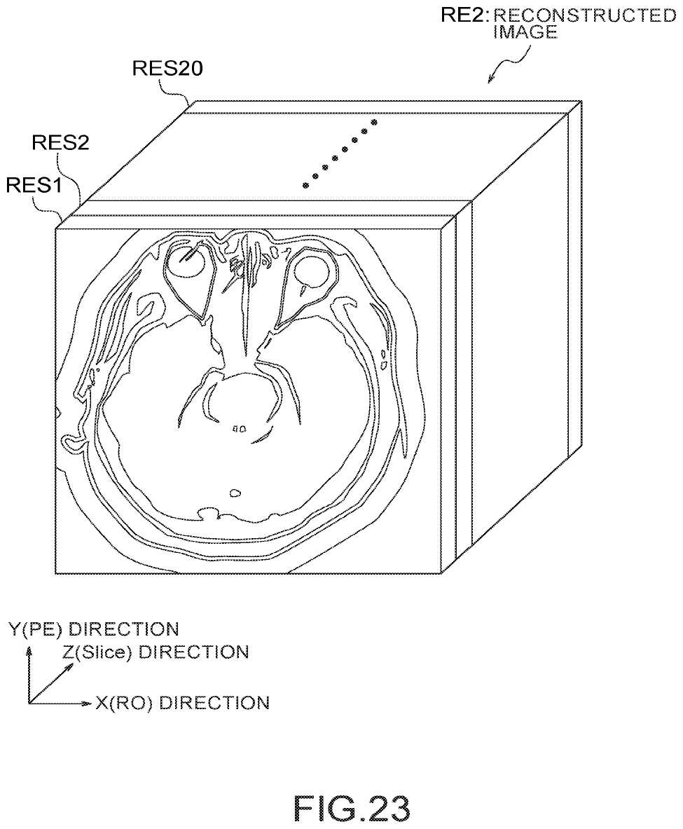

FIG. 23 is a diagram illustrating an example of a reconstructed image generated by the reconstruction processing in the magnetic resonance imaging apparatus 10 according to the present embodiment, and is a diagram corresponding to FIG. 12 in the above-described first embodiment. As illustrated in FIG. 23, in the present embodiment, a reconstructed image RE2 includes a plurality of substantially parallel slices RES1 to RES20 based on the imaging conditions of the locator imaging set in step S49. The slices RES1 to RES20 constitute a three-dimensional reconstructed image RE2 including the region of interest ROI of the subject P. Here, one slice image is an image extending in the X(RO) direction, the Y(PE) direction, and the Z(Slice) direction illustrated in FIG. 23, and the slices RES1 to RES20 are arranged in the Z direction to realize a three-dimensional data structure.

Next, as illustrated in FIG. 15, the magnetic resonance imaging apparatus 10 applies the region of interest ROI to the reconstructed image RE2 (step S53). The processing of applying the region of interest ROI is realized by the output image generation function 1317 in the processing circuitry 131. Specifically, the magnetic resonance imaging apparatus 10 applies the region of interest ROI set by the region-of-interest setting function 1313 in step S45 to the reconstructed image RE2 generated in step S51.

More specifically, the region of interest ROI is applied to the reconstructed image RE2 by cutting out the region of interest ROI set in step S45 from the reconstructed image RE2 generated in step S51. Note that, in the present embodiment, the region of interest ROI is applied to the reconstructed image RE2 by cutting out the region of interest ROI from the reconstructed image RE2, but the method of applying the region of interest ROI to the reconstructed image RE2 is not limited thereto. That is, a method of applying the region of interest ROI to the reconstructed image RE2 is arbitrary, and for example, a luminance value other than the region of interest ROI to the reconstructed image RE2 may be set to 0.

Next, as illustrated in FIG. 15, the magnetic resonance imaging apparatus 10 generates an output image (step S55). The processing of generating the output image is realized by the output image generation function 1317 in the processing circuitry 131. Specifically, the magnetic resonance imaging apparatus 10 generates an output image based on the reconstructed image RE2 generated in step S51, the region of interest ROI set in step S45, and the output image information set in step S47.

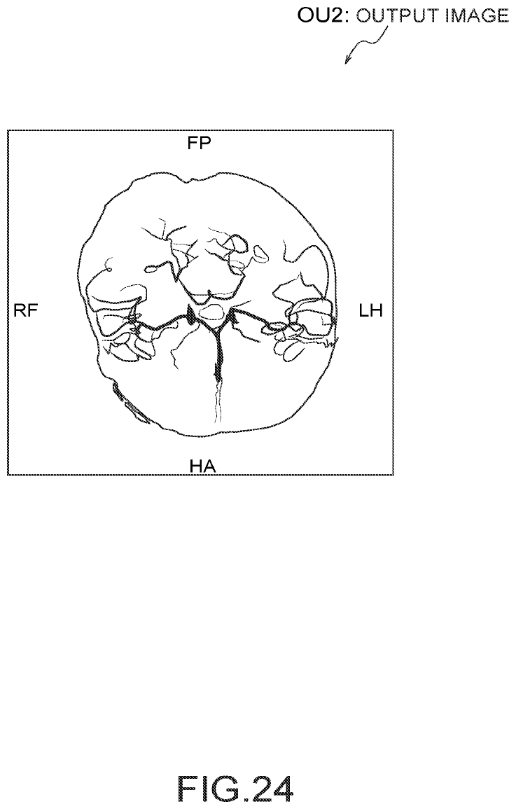

FIG. 24 is a diagram illustrating an example of an output image generated in the magnetic resonance imaging apparatus 10 according to the present embodiment, and is a diagram corresponding to FIG. 14 in the above-described first embodiment. As illustrated in FIG. 24, in the present embodiment, the magnetic resonance imaging apparatus 10 performs the MIP set in step S47 on the reconstructed image RE2 cut out in the region of interest ROI in step S53, and generates the MIP image in which the blood vessel of the brain is emphasized as an output image OU2. Note that the processing in step S33 illustrated in FIG. 15 is equivalent to that in FIG. 2 in the first embodiment described above, and thus description thereof is omitted.

In step S33, the output image OU2 is displayed on the display 134, whereby the output image generation processing according to the present embodiment ends.

As described above, according to the magnetic resonance imaging apparatus 10 of the present embodiment, the magnetic resonance imaging apparatus 10 receives the input operation related to the setting of the region of interest ROI from the operator with respect to the locator image LO2, sets the region of interest ROI according to the received input operation, automatically sets the imaging conditions based on the region of interest ROI, and performs imaging based on the imaging conditions. Therefore, it is possible to shorten the time required for setting the imaging conditions while reducing the burden on the operator. That is, in the present embodiment, the magnetic resonance imaging apparatus 10 receives, from the operator, the input operation for setting a pixel value in the locator image LO2, sets the region of interest ROI, sets the size of the FOV, which is one of the imaging conditions, based on the region of interest ROI such that the FOV of the image imaged in the main imaging includes the region of interest ROI, automatically sets the matrix size, which is one of the imaging conditions, based on the region of interest ROI such that the resolution of the image is higher than the resolution of the locator image LO2, and performs imaging based on the size of the FOV and the matrix size. Therefore, the operator can set the imaging conditions without manually setting the imaging conditions in accordance with the region of interest ROI while referring to the locator image LO2.

Moreover, since the magnetic resonance imaging apparatus 10 sets output image information for generating an output image for the region of interest ROI, generates the reconstructed image RE2 based on the magnetic resonance data collected by imaging, and generates the output image OU2 based on the output image information, the reconstructed image RE2, and the region of interest ROI, it is possible to shorten the time required for generating the output image OU2 while reducing the burden on the operator. That is, in the present embodiment, since the magnetic resonance imaging apparatus 10 sets the three-dimensional image processing to be performed on the reconstructed image as the output image information, cuts out the region of interest ROI from the reconstructed image RE2, and performs the three-dimensional image processing on the reconstructed image RE2 cut out in the region of interest ROI to generate the output image, the operator can generate the output image without creating an image to which the region of interest is manually applied, and manually performing the three-dimensional image processing on the created image.

### First Modification Example According to First Embodiment and Second Embodiment

In the magnetic resonance imaging apparatus 10 according to the first embodiment and the second embodiment described above, by making the matrix size of the image larger than the matrix size of the locator image, it is also possible to automatically set the matrix size, which is one of the imaging conditions, such that the resolution of the image becomes higher than the resolution of the locator image.

### Second Modification Example According to First Embodiment and Second Embodiment

In the magnetic resonance imaging apparatus 10 according to the first embodiment and the second embodiment described above, instead of automatically setting the imaging conditions so that the resolution of the image is higher than the resolution of the locator image, it is also possible to automatically set the imaging conditions by specifying an imaging site based on the locator image, specifying a local site in the imaging site based on the region of interest ROI for the locator image, and reading, from the memory 132, information regarding a preset resolution according to the imaging site and the local site. Hereinafter, a case where this modification example is applied to the first embodiment will be described as a second modification example, and portions different from those of the above-described first embodiment will be described.

In step S23 in the output image generation processing of FIG. 2 described above, the imaging condition setting function 1315 in the processing circuitry 131 specifies an imaging site based on the locator image, specifies a local site in the imaging site based on the region of interest ROI for the locator image, and reads, from the memory 132, information regarding a resolution preset according to the imaging region and the local region, thereby automatically setting the imaging conditions. The information regarding the resolution is information regarding a resolution recommended when imaging a local site according to the imaging site and the local site.

More specifically, in the present modification, using FIG. 7 described above as an example, the imaging condition setting function 1315 analyzes the locator image LO1 illustrated in FIG. 7 to specify that the imaging site is the abdomen from the locator image LO1, and specifies that the local site is the spine from the region of interest ROI set in the locator image LO1. Then, the imaging condition setting function 1315 reads, from the memory 132, the information regarding the resolution in a case where the imaging site is the abdomen and the local site in the imaging site is the spine, and calculates the matrix size based on the information regarding the resolution and the size of the FOV, thereby automatically setting the matrix size, which is one of the imaging conditions.

Note that the information regarding the resolution may include information regarding the matrix size corresponding to the imaging site and the local site. That is, the imaging condition setting function 1315 may automatically set the matrix size, which is one of the imaging conditions, by reading the matrix size, which is the information regarding the resolution, from the memory 132 according to the local site in the imaging site.

As described above, in the magnetic resonance imaging apparatus 10 according to the second modification example, the imaging site is specified based on the locator image LO1, the local site in the imaging site is specified based on the region of interest ROI for the locator image LO1, and the imaging conditions are automatically set by reading, from the memory 132, the information regarding the preset resolution according to the imaging site and the local site. Therefore, it is possible to set an appropriate resolution according to the imaging site and the local site, and it is possible to shorten the time required to set the imaging conditions while reducing the burden on the operator.

Note that, in the second modification example, the imaging condition setting function 1315 may compare the information regarding the resolution read from the memory 132 with the resolution of the locator image LO1, and in a case where the resolution included in the information regarding the resolution read from the memory 132 is higher than the resolution of the locator image LO1, the resolution included in the information regarding the resolution read from the memory 132 may be set as the imaging conditions. As a result, the resolution of the image imaged in the main imaging can be made higher than the resolution of the locator image LO1.

Furthermore, the description of the second modification example described above is a description applied to the first embodiment, but it is obvious that the present modification example can also be applied to the second embodiment.

### Other Modification Examples According to First Embodiment and Second Embodiment

In the magnetic resonance imaging apparatus 10 according to the first embodiment and the second embodiment described above, the output image information setting function 1314 sets MPR as the output image information by the selection of the operator when the region-of-interest setting function 1313 receives, from the operator, the input operation for superimposing a figure indicating the region of interest ROI on the locator image and sets the region of interest ROI, and the output image information setting function 1314 sets MIP as the output image information by the selection of the operator when the region-of-interest setting function 1313 receives, from the operator, the input operation for setting the pixel value and sets the region of interest ROI, but the present invention is not limited thereto. That is, in a case where the region-of-interest setting function 1313 receives, from the operator, an input operation for superimposing a figure indicating the region of interest ROI on the locator image and sets the region of interest ROI, the output image information setting function 1314 may set the MIP as the output image information according to the selection of the operator, and in a case where the region-of-interest setting function 1313 receives, from the operator, an input operation for setting the pixel value and sets the region of interest ROI, the output image information setting function 1314 may set the MPR as the output image information according to the selection of the operator.

Note that the word “processor” used in above descriptions means circuits such as, for example, a Central Processing Unit (CPU), a Graphics Processing Unit (GPU), an Application Specific Integrated Circuit (ASIC), a programmable logic device (for example, a Simple Programmable Logic Apparatus (SPLD), a Complex Programmable Logic Apparatus (CPLD), and a Field Programmable Gate Array (FPGA)). The processor executes functions by reading and executing programs stored in the memory. Note that programs may be configured to be directly integrated in the processor instead of being storing in the memory. In this case, the processor realizes functions by reading and executing programs stored in the circuit. Note that the processor is not limited to the case arranged as a single processor circuit, but may be configured as a single processor by combining a plurality of independent circuits to realize functions. Furthermore, a plurality of component elements may be integrated into one processor to realize the functions.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. A magnetic resonance imaging apparatus comprising:
processing circuitry configured to set a region of interest for a locator image;
automatically set an imaging condition based on the region of interest; and
cause a sequence control circuitry to perform imaging based on the imaging condition, the sequence control circuitry performing the imaging.

2. The magnetic resonance imaging apparatus of claim 1, wherein the processing circuitry is further configured to
set output image information for generating an output image for the region of interest;
generate a reconstructed image based on magnetic resonance data collected by the imaging; and
generate the output image based on the reconstructed image, the region of interest, and the output image information.

3. The magnetic resonance imaging apparatus of claim 1, wherein the processing circuitry is further configured to receive, from an operator, an input operation related to setting of the region of interest, and set the region of interest according to the input operation.

4. The magnetic resonance imaging apparatus of claim 3, wherein the processing circuitry is further configured to receive, from the operator, the input operation for superimposing a figure on the locator image, and set the region of interest, the figure indicating the region of interest.

5. The magnetic resonance imaging apparatus of claim 3, wherein the processing circuitry is further configured to receive, from the operator, the input operation for setting a pixel value in the locator image, and set the region of interest.

6. The magnetic resonance imaging apparatus of claim 1, wherein the processing circuitry is further configured to receive, from an operator, an input operation for adjusting the region of interest, and adjust the region of interest according to the input operation.

7. The magnetic resonance imaging apparatus of claim 1, wherein the processing circuitry is further configured to automatically set the imaging condition such that a field of view (FOV) of an image to be imaged includes the region of interest.

8. The magnetic resonance imaging apparatus of claim 1, wherein the processing circuitry is further configured to automatically set the imaging condition such that a resolution of an image to be imaged becomes higher than the resolution of the locator image.

9. The magnetic resonance imaging apparatus of claim 8, wherein the processing circuitry is further configured to, when a field of view (FOV) of the image to be imaged is smaller than the field of view of the locator image, automatically set the imaging condition such that the resolution of the image to be imaged becomes higher than the resolution of the locator image by making a matrix size of the image to be imaged same as a matrix size of the locator image.

10. The magnetic resonance imaging apparatus of claim 8, wherein the processing circuitry is further configured to automatically set the imaging condition such that the resolution of the image to be imaged becomes higher than the resolution of the locator image by making a matrix size of the image to be imaged larger than a matrix size of the locator image.

11. The magnetic resonance imaging apparatus of claim 1, wherein the processing circuit is further configured to
identify an imaging site based on the locator image,
identify a local site in the imaging site based on the region of interest for the locator image, and
automatically set the imaging condition by reading, from a memory, information regarding the resolution preset according to the imaging site and the local site.

12. The magnetic resonance imaging apparatus of claim 11, wherein the processing circuitry is further configured to
compare the information regarding the resolution read from the memory with the resolution of the locator image, and
when the resolution included in the information regarding the resolution read from the memory is higher than the resolution of the locator image, set the resolution included in the information regarding the resolution read from the memory as the imaging condition.

13. The magnetic resonance imaging apparatus of claim 2, wherein the processing circuitry is further configured to
cut out the region of interest from the reconstructed image, and
generate the output image based on the reconstructed image cut out in the region of interest.

14. The magnetic resonance imaging apparatus of claim 2, wherein the output image is a multi planar reconstruction (MPR) image.

15. The magnetic resonance imaging apparatus of claim 2, wherein the output image is a maximum intensity projection (MIP) image.

16. The magnetic resonance imaging apparatus of claim 5, wherein the processing circuitry is further configured to, based on the pixel value, perform, on the locator image, segmentation processing by threshold processing using the pixel value, and set the region of interest.

17. The magnetic resonance imaging apparatus of claim 14, wherein the MPR image includes at least one of an axial image, a sagittal image, and a coronal image.

18. The magnetic resonance imaging apparatus of claim 2, wherein the processing circuitry is further configured to
display an output image information setting screen for setting the output image information, and
set the output image information by causing a user to select the output image information.

19. The magnetic resonance imaging apparatus of claim 18, wherein the output image information setting screen is a screen in which multi planar reconstruction (MPR) or maximum intensity projection (MIP) can be selected as three-dimensional image processing performed on the reconstructed image.

20. The magnetic resonance imaging apparatus of claim 19, wherein the output image information setting screen is a screen in which at least one cross section of Axial, Sagittal, Coronal, and Radial can be selected in the MPR.
